(19) United States
(12) Patent Application Publication
(10) Pub. No.: US 2018/0161295 A1
(43) Pub. Date: Jun. 14, 2018

(54) TREATMENT OF CLOSED HEAD INJURY AND HEMORRHAGIC STROKE WITH HBED

(71) Applicant: University of Florida Research Foundation, Incorporated, Gainesville, FL (US)

(72) Inventor: Raymond J. Bergeron, JR., Gainesville, FL (US)

(73) Assignee: University of Florida Research Foundation, Incorporated, Gainesville, FL (US)

(21) Appl. No.: 15/536,113

(22) PCT Filed: Dec. 16, 2015
(86) PCT No.: PCT/US15/65985

§ 371 Date: Jun. 14, 2017

Related U.S. Application Data
(60) Provisional application No. 62/092,689, filed on Dec. 16, 2014.

(51) Int. Cl. A61K 31/195; A61K 9/00; A61P 43/00

(52) U.S. Cl. CPC A61K 31/195; A61P 43/00; A61K 9/0019

(57) ABSTRACT

The present invention provides methods for treating closed head injuries or hemorrhagic strokes using a compound of Formula (I), or a pharmaceutically acceptable salt thereof. Compounds of Formula (I) may act as iron chelators to prevent formation of highly reactive radicals leading to additional damage in the closed head injuries or hemorrhagic strokes.

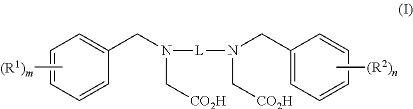

### RELATED APPLICATIONS

This application is a national stage filing under 35 U.S.C. § 371 of international PCT application, PCT/US2015/065985, filed Dec. 16, 2015, which claims priority under 35 U.S.C. § 119(e) to U.S. Provisional Application, U.S. Ser. No. 62/092,689, filed Dec. 16, 2014, each of which is incorporated herein by reference.

### GOVERNMENT SUPPORT

This invention was made with government support under Grant No. DK049108 awarded by the National Institutes of Health. The government has certain rights in the invention.

### BACKGROUND OF THE INVENTION

Closed head injuries refer to a type of brain injury that does not penetrate the skull or dura mater (Ibrahim et al., J. Neurotrauma, 2009, 1021-1035; Fabiani, Italian J. Neurological Sci. 1999, 20(3), 145-153). Closed head injuries are usually caused by blows to the head and frequently occur in traffic accidents, falls, and assaults. Closed head injuries are the leading cause of death in children under 4 years old and the most common cause of physical disability and cognitive impairment in young people. There are several different types of closed head injury, including concussion, brain contusion, diffuse axonal injury, and intracranial hematoma. Intracranial hematomas form when a head injury causes blood vessels to rupture, and blood accumulate within the brain or between the brain and the skull (Teasdale, BMJ, 1990, 300(6721), 363-367).

Hemorrhagic strokes are a type of stroke having bleeding within the brain (intracerebral hemorrhage) and bleeding between the inner and outer layers of the tissue covering the brain (subarachnoid hemorrhage). Hemorrhagic strokes cause an increase in pressure on the brain and lead to death if not timely treated. Therefore, there is a need to develop effective and safe treatment for these neurological diseases.

### SUMMARY OF THE INVENTION

The present invention stems from the recognition that the pathogenesis of closed head injuries and hemorrhagic strokes involve free iron and the generation of reactive oxygen species (ROS), including superoxide anion, hydrogen peroxide, hypochlorous acid, and hydroxyl radicals, and other longer lived, free radicals. Such radicals are now realized to be important contributors to secondary brain damage. As appreciated in the art, free iron contributes to the formation of reactive oxygen species. For example, iron ions in biological systems react with oxygen species to produce highly reactive hydroxyl radicals via the Fenton reaction (see scheme below). The hydroxyl radical is a highly effective oxidizing agent, reacting at a diffusion-controlled rate with most organic species, such as nucleic acids, proteins, and lipids. Furthermore, superoxide anions or a biological reductant (e.g., ascorbic acid) can reduce the resulting Fe⁺³ ion back to Fe⁺² for continued peroxide reduction, thus a problematic cycle.

Fe(II)+H₂O₂ [symbol] Fe(III)+HO^{▪}+HO⁻

Fe(III)+O₂^{▪}− [symbol] Fe(II)+H₂O₂

Therefore, closed head injuries and hemorrhagic strokes involve the possibility that reactive oxygen species will come in contact with iron ions to produce highly reactive and damaging hydroxyl radicals. That is, the iron released from red blood cells react with oxygen species produced by inflammatory cells such as neutrophils to produce hydroxyl radicals that cause cell and tissue injury (Lee, Acta Neurochir Suppl., 2011, 112, 101-106).

N,N′-bis(2-hydroxybenzyl)ethylenediamine-N,N′-diacetic acid (HBED) is a compound known to be an iron chelator and useful as a source of iron in plant nutrition (see U.S. Pat. No. 3,758,540). Bergeron et al. developed HBED and related compounds for treating disease associated with iron overload (see U.S. Pat. Nos. 6,242,492 and 6,531,510).

The present invention stems from the discovery that HBED-related compounds may constitute effective and safe treatment for closed head injuries and/or hemorrhagic strokes. In one aspect, the present invention provides methods of treating and/or preventing a closed head injury by using iron chelators such as compounds of Formula (I):

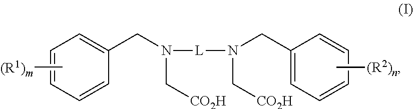

or a pharmaceutically acceptable salt thereof, wherein R¹, R², m, n, and L are as defined herein.

In one aspect, the present invention provides methods of treating and/or preventing a hemorrhagic stroke by using iron chelators such as compounds of Formula (I):

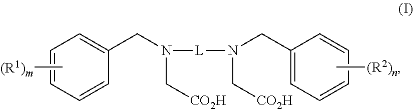

or a pharmaceutically acceptable salt thereof, wherein R¹, R², m, n, and L are as defined herein.

The compounds of Formula (I) may reduce the amount of iron in the tissue (e.g., brain) and thus may prevent formation of toxic highly reactive radicals. In certain embodiments, the compound of Formula (I) is of Formula (II):

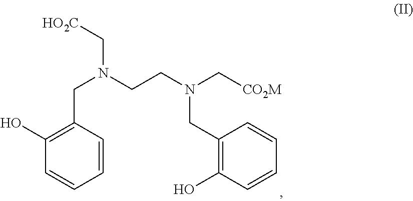

wherein M is a monovalent, pharmaceutically acceptable cation (e.g., Na⁺). In certain embodiments, M is an alkali metal cation such as Na₊ or K₊. In certain embodiments, M is NEₜ⁺.

In certain embodiments, the compound is administered parenterally. In certain embodiments, the compound is administered subcutaneously. In certain embodiments, the compound is administered intravenously.

In another aspect, the present invention provides compounds of Formula (I) or pharmaceutical compositions thereof for use to treat a closed head injury or a hemorrhagic stroke.

The present invention also provides kits with a compound of Formula (I) or pharmaceutical compositions thereof for use in the treatment of closed head injuries or hemorrhagic strokes. Such kits may include one or more unit dosage forms of the compound or pharmaceutical composition to be administered to a subject. In certain embodiments, the kit may include enough unit dosage forms for a course of treatment or for a particular time period (e.g., a week, 10 days, 14 days, a month). The kits may also include packaging information describing the use or prescribing information for the subject or a health care professional. Such information may be required by a regulatory agency such as the U.S. Food and Drug Administration (FDA). The kit may also optionally include a device for administration of the compound or pharmaceutical composition, for example, a syringe for parenteral administration.

The details of one or more embodiments of the invention are set forth in the accompanying Detailed Description, Examples, Claims, and Figures. Other features, objects, and advantages of the invention will be apparent from the description and claims.

The references, web pages, scientific journal articles, patent applications, and issued patents cited in this application are incorporated herein by reference.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR_{cc})R^{bb}, —SH, —SR^{aa}, —SSR^{cc}, —C(═O)R^{aa}, —CO₂H, —CHO, —C(OR_{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(=NR^{bb})R^{aa}, —C(=NR^{bb})OR^{aa}, —OC(=NR^{bb})R^{aa}, —OC(=NR^{bb})OR^{aa}, —C(=NR^{bb})N(R^{bb})₂, —OC(=NR^{bb})N(R^{bb})₂, —NR^{bb}C(=NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO_{2OR}^{aa}, —OSO₂R^{aa}, —S(═O)R^{aa}, —OS(═O)R^{aa}, —Si(R^{aa})₃, —OSi(R^{aa})₃ —C(=S)N(R^{bb})₂, —C(═O)SR^{aa}, —C(=S)SR^{aa}, —SC(=S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR_{cc})₂, —OP(═O)(R^{aa})₂, —OP(═O)(OR_{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR_{cc})₂, —P(R^{cc})₃⁺X³¹ , —P(OR_{cc})₃⁺X⁻, —P(R_{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R_{cc})₄, —OP(OR^{cc})₄, —B(R^{aa})₂, —B (OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R_{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R_{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C_{1-6 alkyl})₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —OO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br^{—}, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)4⁻, BPh4⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO_{2OR}^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R_{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc} , —SO_{2OR}^{cc} , —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C (═O)OR^{aa}) include, but are not limited to, methyl carbamate, ethyl carbamate 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5di-t-butylphenyl)-1methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, pphenylbenzoate-, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

The term “subject,” as used herein, refers to any animal. In certain embodiments, the subject is a mammal. In certain embodiments, the term “subject”, as used herein, refers to a human (e.g., man, woman, or child). The human may be of either sex and may be at any stage of development. In certain embodiments, the subject has been diagnosed with the condition or disease to be treated (e.g., closed head injury or hemorrhagic stroke). In other embodiments, the subject is at risk of developing the condition or disease (e.g., closed head injury or hemorrhagic stroke). In other embodiments, the subject is suspected of having the condition or disease (e.g., closed head injury or hemorrhagic stroke). In certain embodiments, the subject is an experimental animal (e.g., mouse, rat, dog, primate). The experimental animal may be genetically engineered. In certain embodiments, the subject is a domesticated animal (e.g., dog, cat, bird, horse, cow, goat, sheep).

The terms “administer,” “administering,” or “administration,” as used herein refers to implanting, absorbing, ingesting, injecting, or inhaling a compound of Formula (I) or a pharmaceutical composition thereof.

As used herein, the terms “treatment,” “treat,” and “treating” refer to reversing, alleviating, delaying the onset of, or inhibiting the progress of a disease or disorder, or one or more signs or symptoms thereof, described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example to delay or prevent recurrence.

The terms “effective amount” and “therapeutically effective amount,” as used herein, refer to the amount or concentration of an inventive compound, that, when administered to a subject, is effective to at least partially treat a condition from which the subject is suffering (e.g., closed head injury or hemorrhagic stroke).

The term “reactive oxygen species” or “ROS” refers to molecules or ions formed by the incomplete reduction of oxygen. Reactive oxygen species include superoxide anion (O₂^{▪-}), peroxides such as hydrogen peroxide (H₂O₂), hydroxyl radical (HO^{▪}), and hypochlorous acid (HClO). These molecules are typically chemically reactive. Reactive oxygen species may be formed by any number of mechanisms (e.g. enzymatically, by ionizing radiation, by reaction oxygen with a metal). In certain embodiments, the reactive oxygen species are formed by the reduction of oxygen by an iron ion.

The term “closed head injury” refers to any injury to the head that does not penetrate the skull. Closed head injuries may result from falls, blasts, accidents including vehicular accidents, combat, and assaults. Closed head injuries can lead to hemorrhage or brain swelling, which can result in increased intracranial pressure, which can in turn lead to permanent brain damage or even death. Various types of closed head injury include concussions, brain contusions, diffuse axonal injury, and hematomas.

The term “hemorrhagic stroke” refers to bleeding within the brain (intracerebral hemorrhage) and bleeding between the inner and outer layers of the tissue covering the brain (subarachnoid hemorrhage). As used herein, hemorrhagic stroke includes both intracerebral hemorrhage and subarachnoid hemorrhage.

### BRIEF DESCRIPTION OF THE DRAWINGS

FIG. 1 shows the result of velocity dependent ankle torque of hindlimb muscles from the treatment using NaHBED following mTBI (Example 1).

FIG. 2 shows the result of triceps electromyography (EMG) from the treatment using NaHBED following Traumatic Brain Injury (mTBI) (Example 1).

FIG. 3A shows the scheme of velocity dependent ankle torque and EMG Measurements (Bose et al., J. Neurotrauma, 2002, 19, 1229-1305; Bose et al., J. Neurotrauma, 2002, 19, 1231-1249; Bose et al., Front. Physiol. 2012, 3, 258; Bose et al., J. Neurotrauma, 2013, 30, 1177-1191; Thompson et al., 1996, 7, 2273-2276; Wang et al., J. Neurotrauma, 2002, 19, 875-886). FIG. 3B shows an exemplary recording of ankle torque, displacement and EMGs. Ankle torque, displacement and EMGs are simultaneously recorded and time-locked to onset of dorsiflexion. Velocity dependent ankle torque was tested between 49 and 612 degrees/sec dorsiflexion in awake rats.

FIG. 4 shows increased torque amplitude (line B) and increased EMG amplitude (line C) in the TBI animals compared with the normal ones. Line A represents the spring tension recorded by a force transducer (LVDT). Line D represents the neural activity of the triceps surae muscle measured with the transcutaneous EMG electrodes.

FIG. 5 shows the result of the balance performance evaluation from the rotorod test (postRx wk1 & 3) (see Example 1).

FIG. 6 shows the result of gait performance evaluation from the footprints analyses.

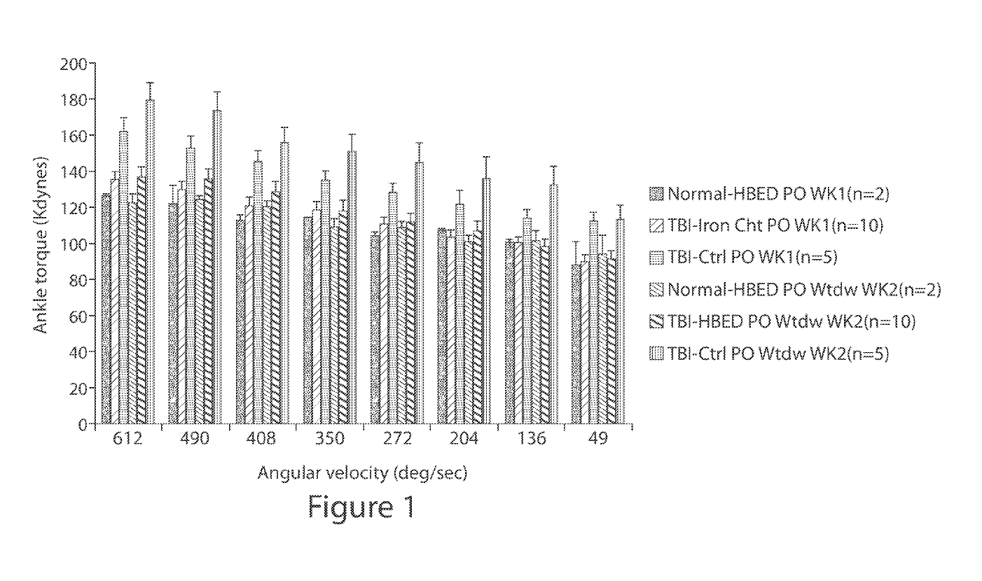

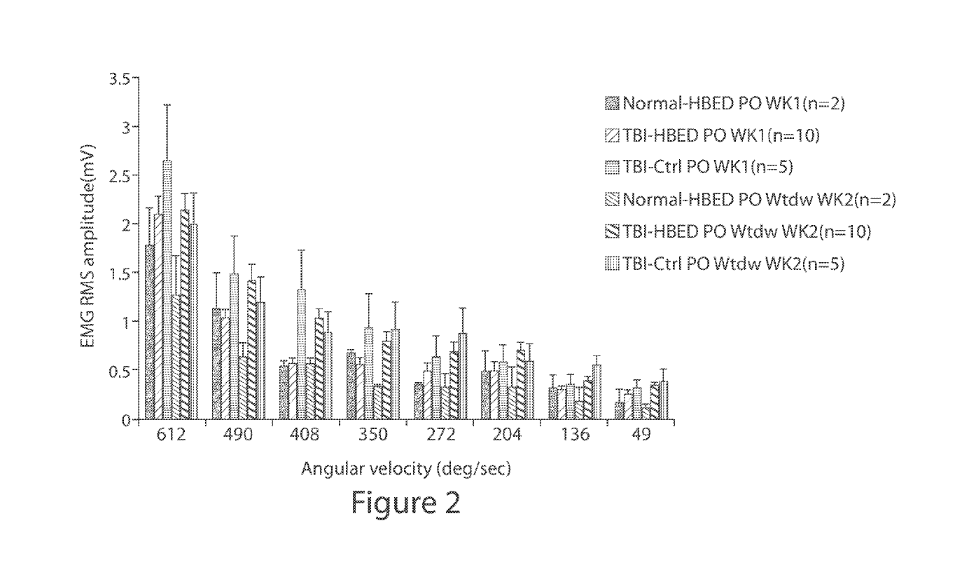

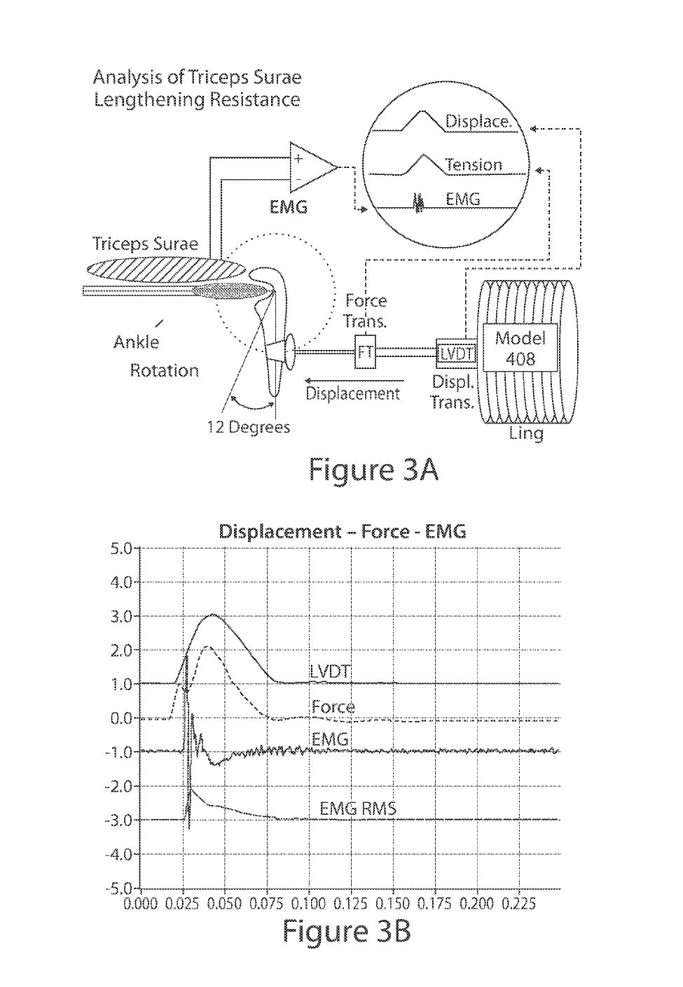

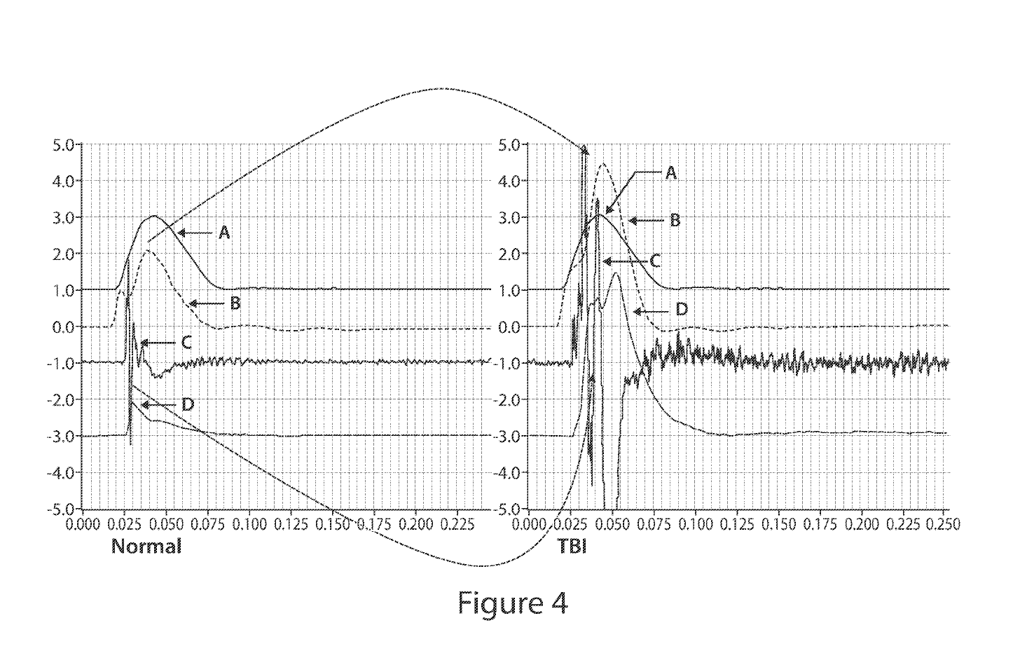

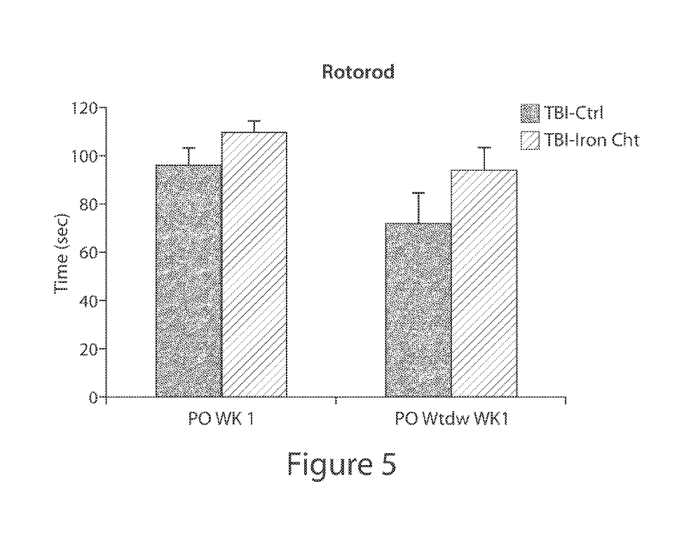

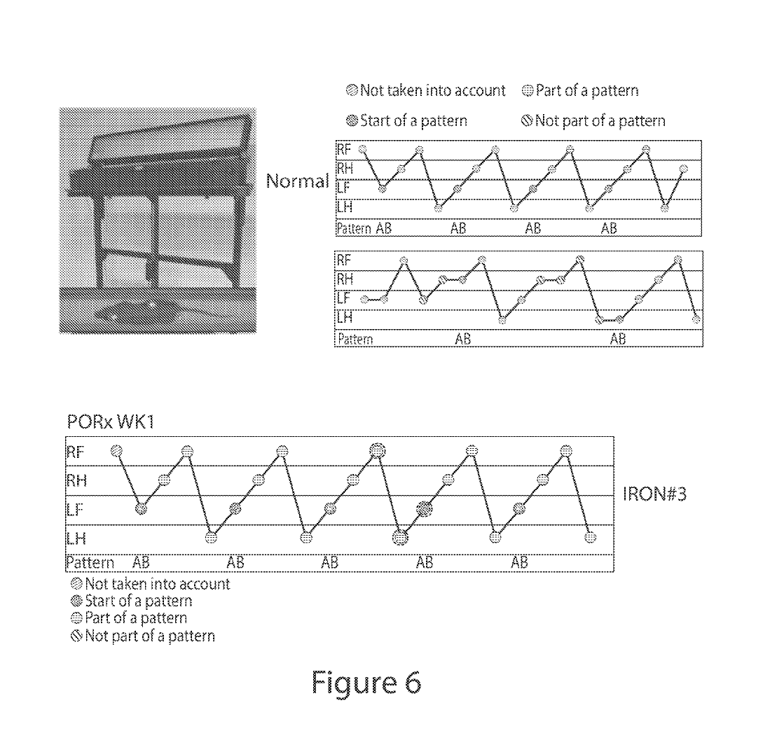

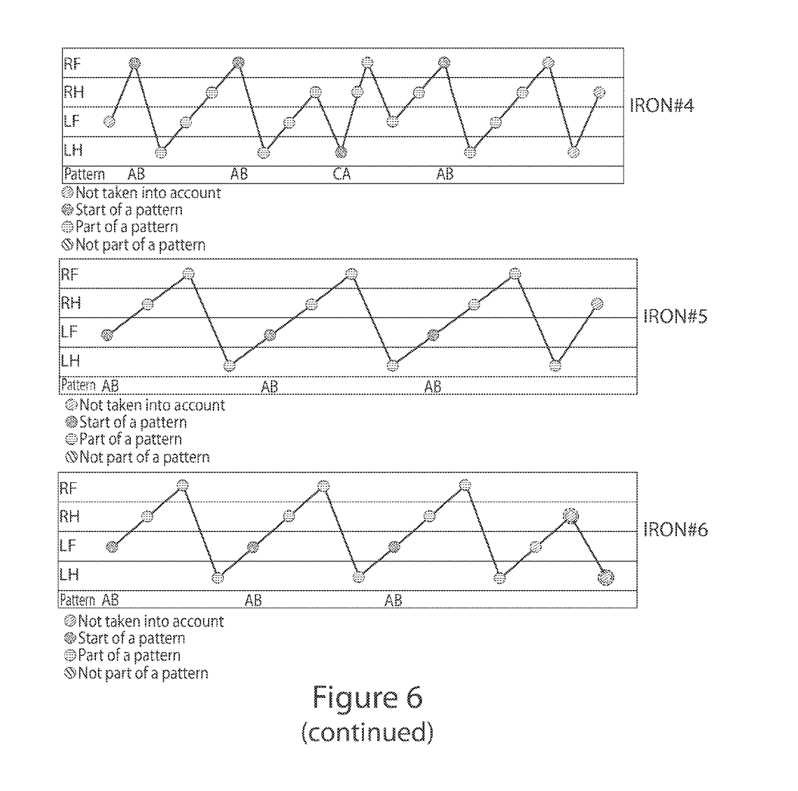

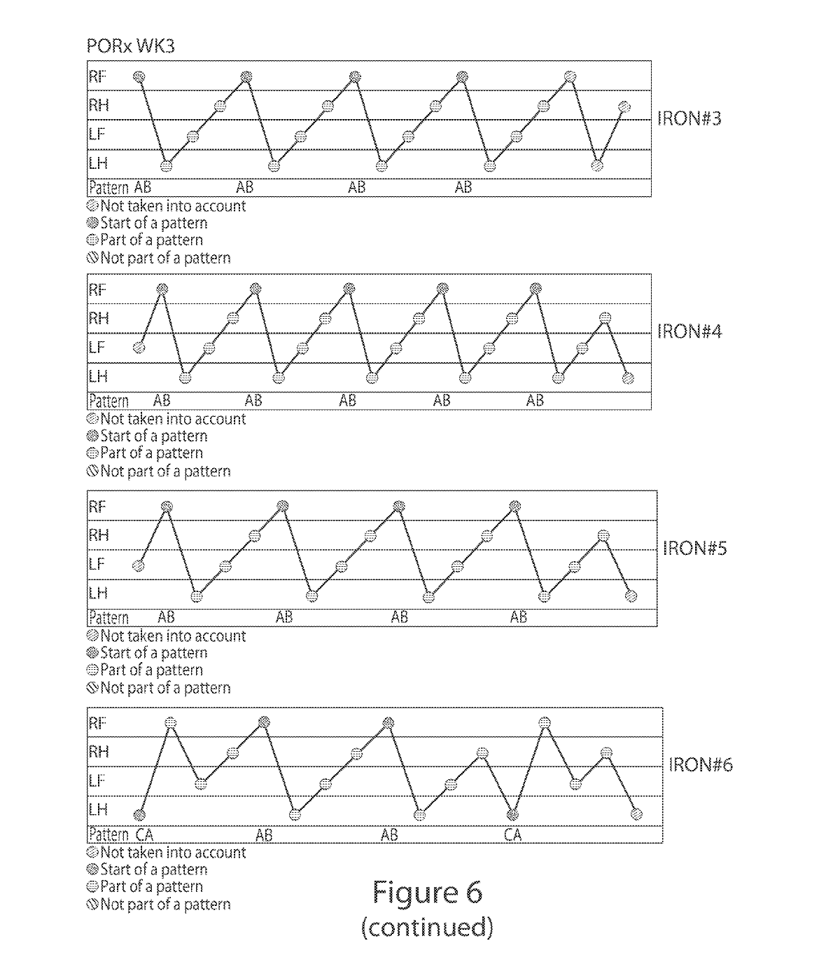

### DETAILED DESCRIPTION OF CERTAIN EMBODIMENTS OF THE INVENTION

Provided herein are compounds, compositions, and methods for treating a closed head injury or a hemorrhagic stroke with an iron chelator such as a compound of Formula (I). When blood vessels are ruptured in a closed head injury or a hemorrhagic stroke, bleeding occurs with hemoglobins released from compromised erythrocytes. The enzyme heme oxygenase can then free iron from hemoglobin. The resulting unmanaged iron ions in the tissues can lead to secondary injury if not timely treated. Without wishing to be bound by any particular theory, the compounds of Formula (I) may chelate iron and may prevent it from participating in the generation of reactive oxygen species. The compounds of Formula (I) may also act as free radical scavengers thereby limiting the damage of reactive oxygen species or other radicals. The invention, therefore, provides methods for treating closed head injuries and hemorrhagic strokes using a compound of Formula (I), pharmaceutical compositions of compounds of Formula (I) for treating closed head injuries and hemorrhagic strokes, or kits.

### Useful Compounds and Pharmaceutical Compositions

Compounds of Formula (I) have been found useful in preventing and treating diseases and conditions associated with iron overload, particularly focal iron overload. Those compounds have been previously described in U.S. Pat. Nos. 4,528,196, 6,242,492 and 6,531,510, each of which is incorporated herein by reference.

The present invention provides compounds of Formula (I)

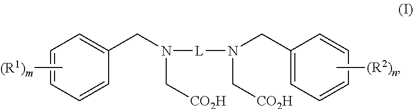

and pharmaceutically acceptable salts thereof, and pharmaceutical compositions thereof, for treating closed head injuries,
wherein:
   each instance of R¹ and R² is independently hydrogen, —CN, —NO₂, halogen, optionally substituted alkyl, —OR^{A}, —N(R^{B})₂, or —CO₂R^{A};
   each occurrence of R^{A} is independently hydrogen, optionally substituted alkyl, or an oxygen protecting group;
   each occurrence of R^{B} is independently hydrogen, optionally substituted alkyl, or an amino protecting group;
   L is an optionally substituted C₁₋₈ alkylene; and
   each of m and n is independently 0, 1, 2, 3, 4, or 5.

In certain embodiments, the present invention provides compounds of Formula (I), and pharmaceutically acceptable salts thereof, for treating closed head injuries.

The present invention provides compounds of Formula (I)

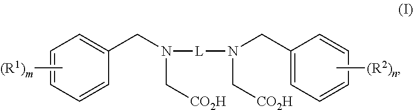

and pharmaceutically acceptable salts thereof, and pharmaceutical compositions thereof, for treating hemorrhagic strokes,
wherein:
   each instance of R¹ and R² is independently hydrogen, —CN, —NO₂, halogen, optionally substituted alkyl, —OR^{A}, —N(R^{B})₂, or —CO₂R^{A};
   each occurrence of R^{A} is independently hydrogen, optionally substituted alkyl, or an oxygen protecting group;
   each occurrence of R^{B} is independently hydrogen, optionally substituted alkyl, or an amino protecting group;
   L is an optionally substituted C₁₋₈ alkylene; and
   each of m and n is independently 0, 1, 2, 3, 4, or 5.

In certain embodiments, the present invention provides compounds of Formula (I), and pharmaceutically acceptable salts thereof, for treating hemorrhagic strokes.

As generally defined herein, R¹ is hydrogen, —CN, —NO₂, halogen, optionally substituted alkyl, —OR^{A}, —N(R^{B})₂, or —CO₂R^{A}. In certain embodiments, R¹ is hydrogen. In certain embodiments, R¹ is halogen. In certain embodiments, R¹ is optionally substituted alkyl. In certain embodiments, R¹ is unsubstituted alkyl. In certain embodiments, R¹ is methyl, ethyl, n-propyl, i-propyl, n-butyl, s-butyl, or t-butyl. In certain embodiments, R¹ is —OR^{A}. In certain embodiments, R¹ is —OH. In certain embodiments, R¹ is —CO₂R^{A}. In certain embodiments, R¹ is —CO₂CH₃.

As generally defined herein, R² is hydrogen, —CN, —NO₂, halogen, optionally substituted alkyl, —OR^{A}, —N(R^{B})₂, or —CO₂R^{A}. In certain embodiments, R² is hydrogen. In certain embodiments, R² is halogen. In certain embodiments, R² is optionally substituted alkyl. In certain embodiments, R² is unsubstituted alkyl. In certain embodiments, R² is methyl, ethyl, n-propyl, i-propyl, n-butyl, s-butyl, or t-butyl. In certain embodiments, R² is —OR^{A}. In certain embodiments, R² is —OH. In certain embodiments, R² is —CO₂R^{A}. In certain embodiments, R² is —CO₂CH₃.

As generally defined herein, L is an optionally substituted C₁₋₈ alkylene. In certain embodiments, L is unsubstituted C₁₋₈ alkylene. In certain embodiments, L is —CH₂—. In certain embodiments, L is —(CH₂)₂—. In certain embodiments, L is —(CH₂)₃—. In certain embodiments, L is substituted C₁₋₈ alkylene. In certain embodiments, L is C₁₋₈ alkylene substituted by an hydroxyl group. In certain embodiments, L is —CH₂CH(OH)CH₂—.

As generally defined herein, R^{A} is independently hydrogen, optionally substituted alkyl, or an oxygen protecting group. In certain embodiments, R^{A} is hydrogen. In certain embodiments, R^{A} is optionally substituted alkyl. In certain embodiments, R^{A} is unsubstituted alkyl (e.g. methyl or ethyl). In certain embodiments, R^{A} is substituted alkyl. In certain embodiments, R^{A} is an oxygen protecting group (e.g. acyl or Boc).

As generally defined herein, R^{B} is independently hydrogen, optionally substituted alkyl, or an amino protecting group. In certain embodiments, R^{B} is hydrogen. In certain embodiments, R^{B} is optionally substituted alkyl. In certain embodiments, R^{B} is unsubstituted alkyl (e.g. methyl or ethyl). In certain embodiments, R^{B} is substituted alkyl. In certain embodiments, R^{B} is a nitrogen protecting group (e.g. acyl or Fmoc).

In certain embodiments, m is 0. In certain embodiments, m is 1. In certain embodiments, m is 2. In certain embodiments, m is 3. In certain embodiments, m is 4. In certain embodiments, m is 5.

In certain embodiments, n is 0. In certain embodiments, n is 1. In certain embodiments, n is 2. In certain embodiments, n is 3. In certain embodiments, n is 4. In certain embodiments, n is 5.

In certain embodiments, the compounds of Formula (I) are of Formula (I-a):

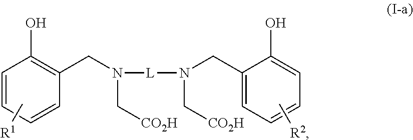

or a pharmaceutically acceptable salt thereof.

In certain embodiments, the compounds of Formula (I) are of Formula (I-a1):

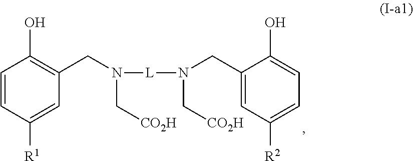

or a pharmaceutically acceptable salt thereof.

In certain embodiments, R¹ and R² are different. In certain embodiments, R¹ and R² are the same. In certain embodiments, both R¹ and R² are hydrogen.

In certain embodiments, the compounds of Formula (I) are of Formula (II):

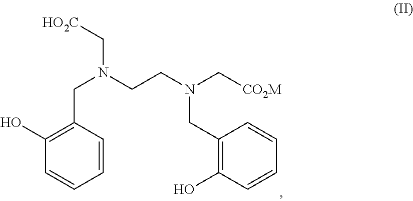

wherein M is a monovalent, pharmaceutically acceptable cation.

As generally defined herein, M is a monovalent, pharmaceutically acceptable cation. The phrase “pharmaceutically acceptable” means that the cation is suitable for administration to a subject. In certain embodiments, M is an inorganic cation. In certain embodiments, M is an alkali metal cation. In certain embodiments, M is Li⁺, Na⁺, K⁺, or Cs⁺. In certain embodiments, M is Na⁺. In certain embodiments, M is K⁺. In certain embodiments, M is NH₄⁺.

Exemplary synthesis of the compounds of Formula (I) can be found in U.S. Pat. Nos. 4,528,196, 6,242,492, and 6,531,510, all of which are incorporated by reference herein.

The present invention provides pharmaceutical compositions comprising a compound of Formula (I), or a pharmaceutically acceptable salt thereof, as described herein, and optionally a pharmaceutically acceptable excipient. In certain embodiments, the pharmaceutical composition of the invention comprises a compound of Formula (I), or a pharmaceutically acceptable salt thereof, and optionally a pharmaceutically acceptable excipient. In certain embodiments, the compound of Formula (I), or a pharmaceutically acceptable salt, is provided in an effective amount in the pharmaceutical composition. In certain embodiments, the effective amount is a therapeutically effective amount. In certain embodiments, the effective amount is a prophylactically effective amount.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage (e.g., therapeutically effective amount or prophylactically effective amount), the pharmaceutical compositions of this invention can be administered to humans and other animals orally, parenterally, intracisternally, intraperitoneally, topically, bucally, or the like, depending on the disease or condition being treated. In certain embodiments, an agent of the invention may be administered orally or parenterally at dosage levels sufficient to deliver from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, a compound of Formula (I) is administered at a dose that is below the dose at which the agent causes non-specific effects.

In certain embodiments, the compound or pharmaceutical composition is administered at a dose of about 0.001 mg to about 3000 mg of compound a day. In certain embodiments, the compound or pharmaceutical composition is administered at a dose of about 0.01 mg to about 2000 mg compound a day. In certain embodiments, the compound or pharmaceutical composition is administered at a dose of about 0.01 mg to about 1000 mg compound a day. In certain embodiments, the compound or pharmaceutical composition is administered at a dose of about 0.1 mg to about 500 mg compound a day. In certain embodiments, the compound or pharmaceutical composition is administered at a dose of about 1 mg to about 100 mg compound a day.

Pharmaceutical compositions described herein can be prepared by any method known in the art of pharmacology. In general, such preparatory methods include the steps of bringing the compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the compounds of Formula (I) and pharmaceutical compositions thereof can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In still another aspect, the present invention also provides a pharmaceutical pack or kit comprising a compound of the invention or a pharmaceutical composition of the invention. In certain embodiments, the kit comprises one or more containers that contain the compound or pharmaceutical composition. In certain embodiments, the kit further includes an additional approved therapeutic agent for use as a combination therapy. Optionally associated with such container(s) can be a notice in the form prescribed by a governmental agency regulating the manufacture, use, or sale of pharmaceutical products, which notice reflects approval by the agency of manufacture, use, or sale for human administration.

### Treatment of Closed Head Injuries

The compounds of Formula (I) and pharmaceutical compositions thereof may be useful in the treatment or prevention of closed head injury, particularly those involving bleeding into the brain or other parts of the central nervous system. Without wishing to be bound by any particular theory, the compounds of Formula (I) may chelate the iron from red blood cells in the blood resulting from the head injury, thereby preventing iron ions from generating reactive oxygen species. In the case of head injury resulting in bleeding into the central nervous system where the vasculature has been compromised a compound being used may or may not have the ability to cross the blood brain barrier. In certain embodiments, the compound being used to treat or prevent a head injury in a subject is not able to cross the blood brain barrier.

Head injuries come in various forms and results from various causes. In certain embodiments, the injury is an injury to the head that penetrates the skull. In other embodiments, the head injury being treated is a closed head injury, which does not penetrate the skull. Closed head injuries result from a variety of causes including accidents, blasts, falls, and assaults, for example, during vehicle accidents, sports activities, or military or combat activities. Types of closed head injuries include concussions, brain contusions, diffuse axonal injury, and hemtoma. In certain embodiments, the closed head injury being treated or prevented in the present invention include closed head injuries that result in blood outside the blood vessels of the brain. The local accumulation of iron from the bleeding is thought to contribute to after effects of closed head injury. By assisting with the clearance of iron from the brain, the effects of the bleeding are minimized.

In certain embodiments, the closed head injury is a concussion. In certain embodiments, the closed head injury is a brain contusion. In certain embodiments, the closed head injury is associated with diffuse axonal injury. In certain embodiments, the closed head injury is an intracranial hematoma. In certain embodiments, the provided methods are used to treat or prevent brain damage of a closed head injury in the sports activities. In certain embodiments, the provided methods are used to treat or prevent brain damage of a closed head injury in the military or combat activities. In certain embodiments, the provided methods are used to treat or prevent brain damage of a closed head injury in an auto-accident.

In the treatment of closed head injury, the compound of Formula (I), or a pharmaceutical composition thereof, may be administered systemically, for example, parenterally or orally. In certain embodiments, the compound or composition is administered orally. In other embodiments, the compound or composition is administered parenterally.

The method has been found to be surprisingly effective when the compound of Formula (I), or pharmaceutical composition thereof, is administered subcutaneously. Subcutaneous administration means that the drug, in the form of an appropriate injectable composition, is injected into the connective tissue just below the skin. The injection may be a formulation, particularly a solution, that provides a controlled release of the compound, but is preferably an aqueous solution. Generally, the subcutaneous administration will be done with excipients that are suitable for subcutaneous administration.

The subject being treated for a head injury may be any type of animal. In certain embodiments, the animal is a mammal. In certain embodiments, the animal is a human. In certain embodiments, the subject is a human child (e.g., infant to 15-year old). In certain embodiments, the animal is a domesticated animal (e.g., dog, cat, pig, cow). In certain embodiments, the animal is a research animal (e.g., mice, rat, dog, primate).

The exact amount of the compound of Formula (I) required to treat a head injury will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dose will be decided by a physician using sound medical judgment. The specific therapeutically effective dose level for any particular subject will depend upon a variety of factors including the severity of the head injury; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the treatment of a head injury in a subject may range from 0.01 mg/kg to 200 mg/kg per unit dosage. In certain embodiments, the unit dosage ranges from 0.1 mg/kg to 100 mg/kg. In certain embodiments, the unit dosage ranges from 0.1 mg/kg to 50 mg/kg. In certain embodiments, the unit dosage ranges from 0.1 mg/kg to 20 mg/kg. In certain embodiments, the unit dosage ranges from 0.1 mg/kg to 10 mg/kg. In certain embodiments, the unit dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound of a composition thereof is administered every other day, every third day, every week, every other week, or every month. In certain embodiments, the inventive treatment is stopped once the head injury is resolved, or it is thought the inventive treatment would no longer be beneficial. In certain embodiments, the treatment is stopped once the bleeding has been resolved in a subject with a head injury.

In certain embodiments for treatment of closed head injury, the daily dosage is about 0.001 milligram to 1000 milligrams per kilogram of the subject's body weight. In certain embodiments for treatment of closed head injury, the daily dosage is about 1.0 milligram to 500 milligrams per kilogram of the subject's body weight. In certain embodiments for treatment of closed head injury, the daily dosage is about 5.0 milligram to 500 milligrams per kilogram of the subject's body weight. In certain embodiments for treatment of closed head injury, the daily dosage is about 5.0 milligram to 200 milligrams per kilogram of the subject's body weight. In certain embodiments, the daily dosage is about 5.0 milligram to 100 milligrams per kilogram of the subject's body weight. In certain embodiments, the daily dosage is about 5.0 milligram to 50 milligrams per kilogram of the subject's body weight.

In certain embodiments, the treatment lasts from one day to about three months. In certain embodiments, the treatment lasts from one day to about two months. In certain embodiments, the treatment lasts from one day to about one months. In certain embodiments, the treatment lasts less than 25 days. In certain embodiments, the treatment lasts less than 20 days. In certain embodiments, the treatment lasts less than 15 days. In certain embodiments, the treatment lasts less than 10 days. In certain embodiments, the treatment lasts less than 5 days.

### Treatment of Hemorrhagic Stroke

The present invention also provides for the treatment or prevention of hemorrhagic stroke. In the treatment of a hemorrhagic stroke, a compound of Formula (I), or composition thereof, may be administered to a subject to prevent or minimize the damage due to reperfusion injury after the blood supply to the affected area of the brain is restored. The compounds may prevent the generation of reactive oxygen species by either chelating iron responsible for the generation of such species and/or quenching such radical species when they do occur.

In certain embodiments, the compounds being used in the treatment may not have the ability to cross the blood brain barrier.

The present invention may be useful in treating a subject after the subject has been diagnosed with having a stroke, or a subject who is susceptible to having a stroke may be administered a compound of Formula (I), or composition thereof, to prevent or minimize the stroke's effects. In certain embodiments, the compound is administered as quickly as possible after a subject has been diagnosed with having a stroke. In certain embodiments, the compound is administered to the subject while the stroke is still occurring. In certain embodiments, the compound, or a composition thereof, is administered to a subject who has a history of strokes or is susceptible to having a stroke because of the subject's underlying medical condition. The compound or composition thereof may be administered once or multiple times in the treatment of stroke.

In the treatment of hemorrhagic stroke, the compound of Formula (I), or a pharmaceutical composition, thereof may be administered systemically, for example, parenterally or orally. In certain embodiments, the compound or composition is administered orally. In other embodiments, the compound or composition is administered parenterally. In other embodiments, the compound or composition is administered subcutaneously.

The provided method has been found to be surprisingly effective when the compound of Formula (I) or pharmaceutical composition thereof is administered subcutaneously. Subcutaneous administration means that the drug, in the form of an appropriate injectable composition, is injected into the connective tissue just below the skin. The injection may be a formulation, particularly a solution, that provides controlled release of the active entity, but is preferably an aqueous solution. Generally, the subcutaneous administration will be done with excipients that are suitable for subcutaneous administration.

In other embodiments, the compound or composition is administered intravenously. The intravenous administration achieves quick administration of the compound or composition as described herein. In certain embodiments, the intravenous route is used for post operation administration.

The subject being treated for hemorrhagic stroke may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate).

The exact amount of the compound of Formula (I) required to treat a hemorrhagic stroke will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dose will be decided by a physician using sound medical judgment. The specific therapeutically effective dose level for any particular subject will depend upon a variety of factors including the severity of the stroke; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the treatment of a stroke in a subject may range from 0.01 mg/kg to 200 mg/kg per unit dosage. In certain embodiments, the unit dosage ranges from 0.1 mg/kg to 100 mg/kg. In certain embodiments, the unit dosage ranges from 0.1 mg/kg to 50 mg/kg. In certain embodiments, the unit dosage ranges from 0.1 mg/kg to 20 mg/kg. In certain embodiments, the unit dosage ranges from 0.1 mg/kg to 10 mg/kg. In certain embodiments, the unit dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound or a composition thereof is administered every other day, every third day, every week, every other week, or every month. Typically the compound or composition thereof is not administered after it is no longer thought to be beneficial, for example, when all the bleeding has been cleared in a hemorrhagic stroke.

In certain embodiments for treatment of hemorrhagic strokes, the daily dosage is about 0.001 milligram to 1000 milligrams per kilogram of the subject's body weight. In certain embodiments for treatment of hemorrhagic strokes, the daily dosage is about 1.0 milligram to 500 milligrams per kilogram of the subject's body weight. In certain embodiments for treatment of hemorrhagic strokes, the daily dosage is about 5.0 milligram to 500 milligrams per kilogram of the subject's body weight. In certain embodiments for treatment of hemorrhagic strokes, the daily dosage is about 5.0 milligram to 200 milligrams per kilogram of the subject's body weight. In certain embodiments, the daily dosage is about 5.0 milligram to 100 milligrams per kilogram of the subject's body weight. In certain embodiments, the daily dosage is about 5.0 milligram to 50 milligrams per kilogram of the subject's body weight.

In certain embodiments, the treatment lasts from one day to about three months. In certain embodiments, the treatment lasts from one day to about two months. In certain embodiments, the treatment lasts from one day to about one months. In certain embodiments, the treatment lasts less than 25 days. In certain embodiments, the treatment lasts less than 20 days. In certain embodiments, the treatment lasts less than 15 days. In certain embodiments, the treatment lasts less than 10 days. In certain embodiments, the treatment lasts less than 5 days.

### EXAMPLES

In order that the invention described herein may be more fully understood, the following examples are set forth. It should be understood that these examples are for illustrative purposes only and are not to be construed as limiting this invention in any manner.

### Example 1

These experiments were performed to assess the influence of treatment using NaHBED following mTBI. Fifteen mTBIs were performed as described above, using 450×1.25m weight drop closed head injury. The animals were randomly divided into two groups, control (n=5) and iron chelator (IC) NaHBED treatment (n=10). Beginning on the first day following injury, NaHBED was delivered by subcutaneous injection of 50 mg/kg, 2× per day, for a total dosage of 100 mg/kg/body weight/day. NaHBED 60 mg/ml, was dissolved in deionized, sterilized, iron-free water. The IC was administered every day for 2 weeks. The control animals received equal volume subcutaneous injections of saline.

Spasticity: The influence of treatment was tested by analysis of lower limb spasticity (velocity dependent ankle torque and triceps EMG), in mTBI treated and non-treated mTBI animals. A normal ICT group (Normal HBED) was also included as a treatment control. The robust velocity dependent spasticity that was recorded in the TBI animals at postinjury (PO) week-1—did not decrease in magnitude, compared to the TBI Ctrl Wk2—indicating an enduring, robust lower limb spasticity. Comparing the lower limb spasticity at PO Wk-1 (1 week of treatment) and at PO Wk-3 (one week following 2 weeks of IC treatment), the IC treated animals reveled significantly lower amplitudes of velocity dependent ankle torques and time locked EMGs.

### Example 2

28-Day Intravenous Repeat Dose Toxicity Study of NaHBED in Male and Female Dogs SRI Study.

Male and female beagle dogs were either infused biweekly with iron dextran to a level of approximately 500 mg iron/kg body weight or held without iron loading during the 2-month pretreatment phase. All dogs were held an additional 2 ½ months before beginning dose administration with N,N′-di(2-hydroxybenzypethylenediarnine-N,N′-diacetic acid, monosodium salt (NaHBED). Groups of 4 male and 4 female dogs that had been iron-loaded received NaHBED at 21.46, 64.38, or 128.76 mg/kg daily in 15-35 min intravenous (i.v.) infusions for 28 consecutive days; a non-iron-loaded group containing the same number of dogs of each sex received 21.46 mg NaHBED/kg/day i.v. during the same period. Vehicle-only control groups of iron-loaded and non-iron-loaded dogs (3 males and 3 females per group) were subjected to the same regimen concurrently. One male and one female each from the 128.76 mg/kg and vehicle only iron-loaded groups were held for an additional 27 days without further treatment to assess recovery from any observed adverse effects. During the treatment period, clinical observations on the animals were made daily after dosing. Body weights were measured before the start of dosing with NaHBED, weekly during the 28-day treatment period, and at study termination when recovery animals were sacrificed; food consumption was measured weekly during the treatment period. Hematology and clinical chemistry parameters, including serum iron parameters, were assessed at Weeks-1, 2, 4, and 8. Urinalysis was performed on bladder urine samples at necropsy. Plasma samples were collected for drug level analysis at selected timepoints after dosing on the first day and 28 days later. Routine ophthalmologic examinations and cardiovascular evaluations were conducted during the week prior to dose administration and during the third and fourth (eye exams) or fourth (cardiology exams) weeks of NaHBED administration. Organ weights were measured at necropsy and organ-to-body and -brain ratios determined. Gross and microscopic examination of a complete set of tissues was performed on iron-loaded high dose (128.76 mg/kg/day) and control groups and on non-iron-loaded low dose (21.46 mg/kg/day) and control groups. Specimens from liver, heart, spleen, lung, kidney, and eye were taken from each dog at necropsy for subsequent tissue analyses of iron and zinc content.

The only observable adverse effects of NaHBED administration in iron-loaded dogs were emesis and/or diarrhea in animals at the high dose level. NaHBED produced no distinguishable effects on body weight and food consumption; ophthalmologic, cardiovascular, hematology, or clinical chemistry and urinalysis parameters; or organ weights or weight ratios. In addition, no histopathologic findings were found that were clearly associated with NaHBED administration. Mean kidney iron content in the iron-loaded male groups and zinc content in the high dose level group were statistically lower. However, although noted, the differences lacked a clear dose relationship to NaHBED and could alternatively be attributed to the small group sizes, and the biological significance was obscure because there were no histopathological findings in the kidneys. Reddish urine was observed throughout most of the treatment period with the majority of dogs at all dose levels but not with the vehicle-only controls; the coloration was attributed to enhanced excretion of iron from the body facilitated by NaHBED.

The most notable effect of NaHBED administration on normal (non-iron-loaded) dogs was a dramatic decrease in the iron and zinc content of kidneys measured at necropsy; females appeared to be more susceptible, showing a virtual absence of either ion in this organ. In addition, there was occasionally a slightly red coloration in the urine of dogs in this dose group and an apparent elevation (statistically significant for males on Day 15) in serum iron and iron-to-transferrin ratio. These observations collectively suggest that NaHBED induced mobilization and enhanced excretion of iron and zinc at the 21.46 mg/kg/day dose level, the only dose level studied in normal dogs. No other effects were seen in this group.

Toxicokinetic parameters were similar in iron-loaded and non-iron-loaded dogs given the same dose level of NaHBED. However, changes in several parameters were noted on Day 28 relative to Day 1. Peak plasma levels (Cₘₐₓ) and area-under-the curve (AUC) in each group were found to be 50% to 70% lower on Day 28, whereas total body clearance and volume of distribution were correspondingly elevated, suggesting that repeated administration of the test agent had resulted in changes in the distribution of the drug.

The results of this study indicate that the no observable adverse effect level (NOAEL) of NaHBED administered in repeated daily i.v. doses to iron-loaded beagle dogs for 28 days is 64.38 mg/kg/day and that the maximum tolerated dose (MTD) level is >128.76 mg/kg/day under these experimental conditions. There is a clear indication of NaHBED-induced mobilization and excretion of iron and zinc at the 21.46 mg/kg/day dose level in non-iron-loaded animals

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating or preventing a closed head injury in a subject comprising administering to the subject a therapeutically effective amount of a compound of Formula (I): 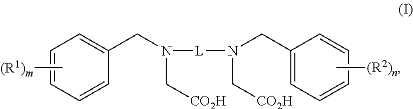
or a pharmaceutically acceptable salt thereof, or a pharmaceutical composition thereof, wherein:
each instance of R¹ and R² is independently hydrogen, —CN, —NO₂, halogen, optionally substituted alkyl, —OR^{A}, —N(R^{B})₂, or —CO₂R^{A};
each occurrence of R^{A} is independently hydrogen, optionally substituted alkyl, or an oxygen protecting group;
each occurrence of R^{B} is independently hydrogen, optionally substituted alkyl, or an amino protecting group;
L is an optionally substituted C₁₋₈ alkylene; and
each of m and n is independently 0, 1, 2, 3, 4, or 5.

2. The method of claim 1, wherein the closed head injury is a concussion, brain contusion, associated with diffuse axonal injury, or is an intracranial hematoma.

3-5. (canceled)

6. A method of treating or preventing a hemorrhagic stroke in a subject comprising administering to the subject a therapeutically effective amount of a compound of Formula (I): 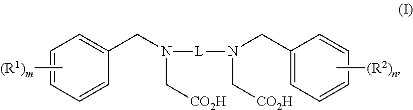
or a pharmaceutically acceptable salt thereof, or a pharmaceutical composition thereof, wherein:
each instance of R¹ and R² is independently hydrogen, —CN, —NO₂, halogen, optionally substituted alkyl, —OR^{A}, —N(R^{B})₂, or —CO₂R^{A};
each occurrence of R^{A} is independently hydrogen, optionally substituted alkyl, or an oxygen protecting group;
each occurrence of R^{B} is independently hydrogen, optionally substituted alkyl, or an amino protecting group;
L is an optionally substituted C₁₋₈ alkylene; and
each of m and n is independently 0, 1, 2, 3, 4, or 5.

7. The method of claim 6, wherein the hemorrhagic stroke is intracerebral hemorrhage or subarachnoid hemorrhage.

8. (canceled)

9. The method of claim 1, wherein the compound is of Formula (I-a): 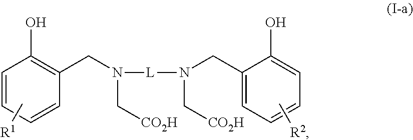
or a pharmaceutically acceptable salt thereof.

10. The method of claim 9, wherein the compound is of Formula (I-a1): 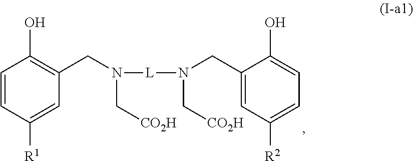
or a pharmaceutically acceptable salt thereof.

11. The method of claim 1, wherein R¹ is hydrogen.

12. The method of claim 1, wherein R² is hydrogen.

13. The method of claim 1, wherein L is unsubstituted C₁₋₈ alkylene.

14. The method of claim 13, wherein L is —CH₂—CH₂— or —CH₂—CH₂—CH₂.

15. (canceled)

16. The method of claim 1, wherein L is substituted C₁₋₈ alkylene.

17. (canceled)

18. The method of claim 1, wherein the compound is of Formula (II): 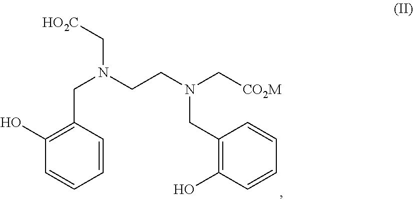
wherein M is a monovalent, pharmaceutically acceptable cation.

19. The method of claim 18, wherein M is an inorganic cation.

20. The method of claim 19, wherein M is an alkali metal cation.

21-23. (canceled)

24. The method of claim 1, wherein the compound or pharmaceutical composition is dissolved in a liquid.

25-27. (canceled)

28. The method of claim 1, wherein the compound or pharmaceutical composition is administered about 0.001 mg to about 3000 mg a day.

29-30. (canceled)

31. The method of claim 1, wherein the daily dosage is about 0.001 milligram to 200 milligrams per kilogram of the subject's body weight.

32. (canceled)

33. The method of claim 1, wherein the compound or the pharmaceutical composition is administered parenterally.

34. The method of claim 33, wherein the compound or the pharmaceutical composition is administered subcutaneously.

35. The method of claim 1, wherein the subject is a mammal.

36-43. (canceled)
